(19) United States
(12) Patent Application Publication
(10) Pub. No.: US 2017/0080124 A1
(43) Pub. Date: Mar. 23, 2017

(54) BIOCOMPATIBLE COMPOSITES AND METHODS OF MAKING SAME

(71) Applicant: REGENTS OF THE UNIVERSITY OF MINNESOTA, MINNEAPOLIS, MN (US)

(72) Inventors: Allison Hubel, Saint Paul, MN (US); Michael Dominick DiVito, Chicago, IL (US); Andreas Stein, Saint Paul, MN (US)

(21) Appl. No.: 15/267,504

(22) Filed: Sep. 16, 2016

Related U.S. Application Data
(60) Provisional application No. 62/220,103, filed on Sep. 17, 2015.

(51) Int. Cl. A61L 27/24; A61L 27/30; A61L 27/18; A61L 27/52

(52) U.S. Cl. CPC A61L 27/24; A61L 27/52; A61L 27/306; A61L 27/18; A61L 2430/16

(57) ABSTRACT

This disclosure describes biocompatible composites and method for making the biocompatible composites. Generally, the biocompatible composite includes a fibril prepared from a biocompatible polymer and cationic component, and a uniform coating of silica-containing material.

### CROSS-REFERENCE TO RELATED APPLICATION

This application claims priority to U.S. Provisional Patent Application No. 62/220,103, filed Sep. 17, 2015, which is incorporated herein by reference.

### SUMMARY

This disclosure describes, in one aspect, a biocompatible composite. Generally, the biocompatible composite includes a fibril that includes a biocompatible polymer and a cationic component and a uniform coating of silica-containing material.

In some embodiments, the uniform coating includes no silica aggregates greater than 100 nm in diameter.

In some embodiments, the biocompatible polymer can include collagen.

In some embodiments, the cationic component can include poly-L-lysine.

In some embodiments, the silica-containing component can include tetraethylorthosilicate (TEOS).

In another aspect, this disclosure describes a method of making a biocompatible composite. Generally, the method includes forming a fibril that includes a hydrogel including a biocompatible polymer, treating the fibril with a cationic component and coating the fibril with a silica-containing component.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. SEM images of: (A) control collagen hydrogel; silica-collagen hydrogels with poly-L-lysine after (B) one day, (D) two days, or (F) three days (bottom right) of TEOS soak (R=4000); and without poly-L-lysine after 1 day soak in (C) R=4000 (middle left) and (E) R=400 (bottom left) TEOS bath. Asterisk marks large silica aggregate. Each white scale bar=1 μm in length.

FIG. 2. SEM image of 50 mg/mL hydrogel before (left) and after (right) poly-L-lysine treatment and three-day TEOS soak. The inset is of the same sample at a higher magnification, where the scale bar in the bottom right is 1 μm long and the inset scale bar is 50 nm long.

FIG. 3. Raman spectra of (a) collagen, (b) +PLL, (c) +TEOS, (d) TA10, and (e) TA10-UV4. The dotted lines highlight the change and shift of the silica peak between the dotted lines.

FIG. 4. Collagenase degradation measured in terms of sample mass versus time of silica-collagen hydrogels (50 mg/mL collagen concentration). Error bars represent confidence intervals (n=4).

FIG. 5. Spectral transmittance of control and silica-collagen hydrogel in comparison to published results for excised rabbit cornea (McLaren J W and Brubaker R F, 1996. Curr Eye Res 15(4):411-421). The inset contains images of the control sample (left) and silica-collagen hydrogel (right) on top of a ruler.

FIG. 6. Relaxed moduli for different collagen concentrations and silica deposition treatments. 5 mg/mL: soaked in LUDOX (W. R. Grace & Co., Columbia, Md.) for one hour (LUDOX 0), followed by aging in TEOS solution for one day (LUDOX 1) or for three days (LUDOX 3). Error bars represent confidence intervals (n=4).

### DETAILED DESCRIPTION OF ILLUSTRATIVE EMBODIMENTS

The cornea is a commonly transplanted tissue in the United States. The supply of natural donor tissue is typically insufficient to meet demand. The cornea is a unique tissue that requires a stiff, elastic scaffold to be biocompatible and highly transmissive to visible light. Highly concentrated collagen hydrogels (>50 mg/mL) made in vitro approach these design requirements, but degrade rapidly in physiological conditions.

Sol gel chemistry has been used to prepare silica-collagen hydrogels with improved mechanical properties. Silica gels derived from the sol gel process can exhibit controllable biodegradation behavior. Silica-collagen xerogel and hydrogel materials can withstand implantation on explanted rabbit corneas and allow re-epithelializaton, which is important for integration with the native tissue. Also, copolymerization methodology when making silica-collagen hydrogels can involve mixing silica sol and collagen in their liquid states, which can be problematic with highly concentrated and more viscous collagen solutions.

In contrast, this disclosure describes a two-step process in which a collagen hydrogel is formed followed by silica deposition onto previously formed collagen fibrils. In order to strengthen the collagen fibril network with silica without reducing light transmission, a controlled sol gel process that limits silica condensation to a smooth, uniform coating around the collagen fibrils was developed. This disclosure characterizes the microstructures produced by this new method of silica-collagen hydrogel synthesis. Macroscopic biomechanical and optical characteristics of the nanocomposite material are also determined. Finally, the effects of different aging conditions on chemical bonding of the silica network are characterized and the degradation of the material in the presence of collagenase quantified. This novel method of constructing a silica-collagen nanocomposite permits modulation of the biomechanical, optical, and/or degradation properties of the composite and therefore represents an important candidate material for corneal replacement.

### Microstructure

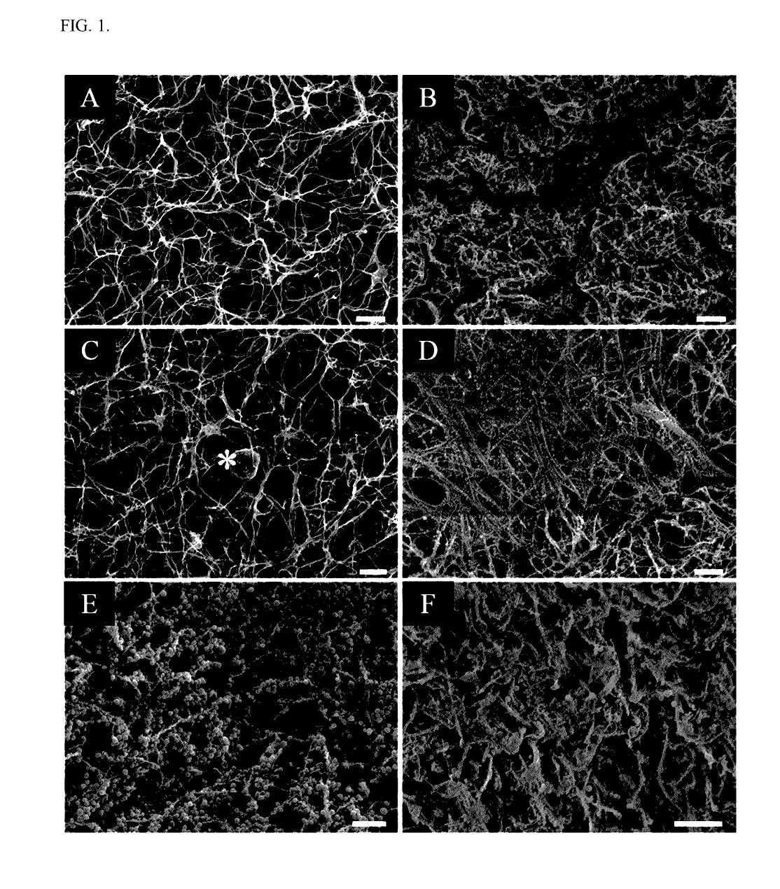

A dilute collagen concentration of 5 mg/mL was used to show the influence of poly-L-lysine as an axemplary cationic component on the formation of the nanocomposite (silica/collagen). FIG. 1(C) illustrates that in the absence of poly-L-lysine surface modification, silica forms large (>1 μm) aggregrates, leaving the majority of collagen fibril surfaces exposed. However, when hydrogels are pretreated with poly-L-lysine, small silica aggregration (<100 nm) occurs preferentially at the surface of collagen fibrils (FIG. 1B, FIG. 1D). These experiments show that the presence of silica at the fibril surface affects the fixation and drying process of the gel, which causes collapse of the network and fibril bundling (FIG. 1D, FIG. 1F). However, in poly-L-lysine-treated samples there is silica at the fibril surface and an absence of large homogeneous silica aggregrates.

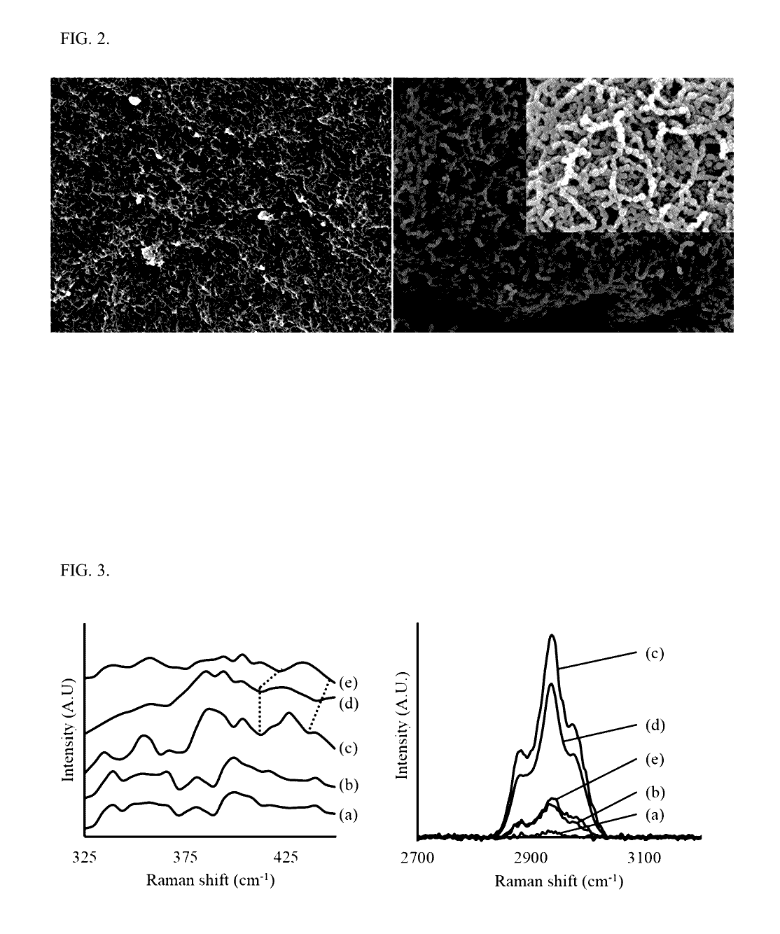

At higher collagen concentrations (e.g., 50 mg/mL), a uniform silica coating was observed. FIG. 2 compares the microstructure of 50 mg/mL pure collagen hydrogel to that of hydrogels that were pretreated with poly-L-lysine and soaked in Stöber solution for three days. After silica deposition, there was a statistically significant increase in fibril diameter from 18.5±0.7 to 48.9±1.3 nm (n=100 measurements for each nominal value).

### Chemical Composition

Raman spectroscopy was performed to characterize the chemical composition and bonding present at different points of the silica-collagen hydrogel synthesis. Sample (b) of FIG. 3 shows that after soaking collagen in poly-L-lysine solution, there is a significant amplitude increase of peaks centered about 2940 cm⁻¹, which are associated with vibrations of —CH bonds. The increase in peak amplitude at 2940 cm⁻¹ suggests that poly-L-lysine is present even after rinsing and removal of solvent from the hydrogel. Upon soaking the material in Stöber solution, a sharp peak appears at approximately 430 cm⁻¹ (FIG. 3, sample (c)), associated with siloxane bonds (Si—O—Si) bending. Additionally, an increase in —CH bonds is present, which is likely from alkyl side chains of TEOS that remain unhydrolyzed. After aging and UV exposure (FIG. 3, sample (e)), the decrease in intensity of the peak related to —CH bonds indicates a higher degree of TEOS hydrolysis, while the broadening and shift to a higher frequency of the siloxane peak indicates an increase in condensation of the silica network.

### Collagenase Degradation Resistance

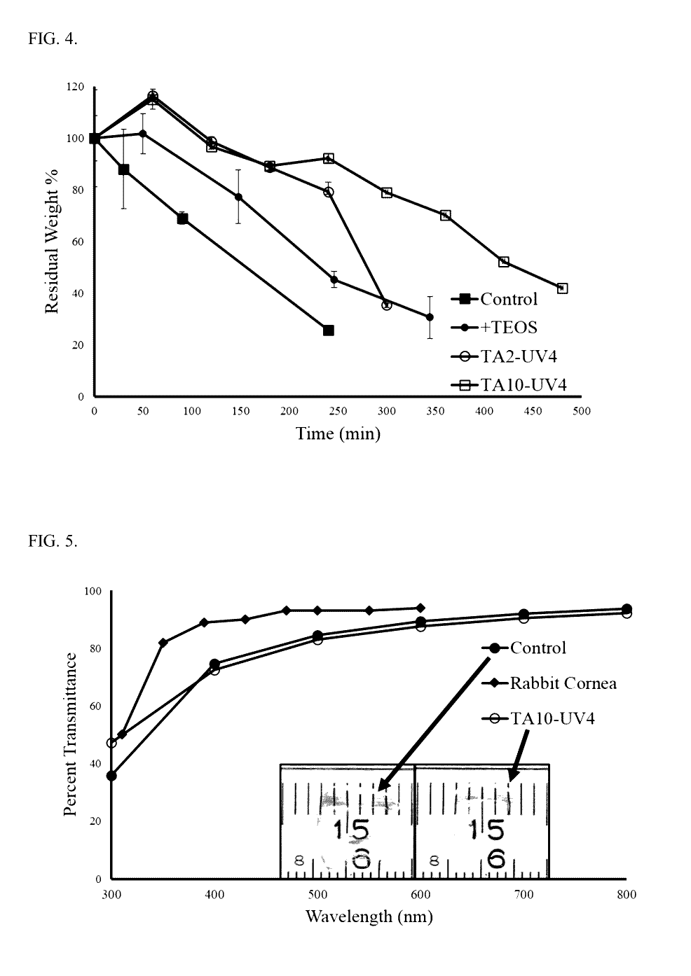

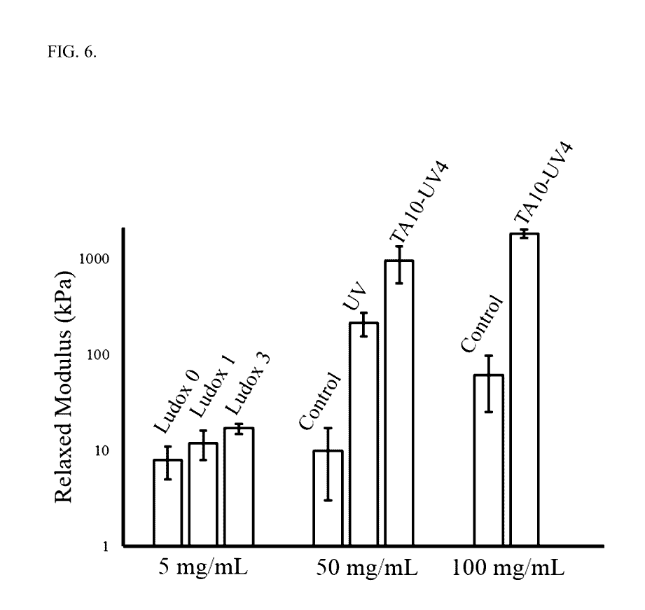

Unmodified collagen gels can be sensitive to collagenase degradation. The deposition of silica using poly-L-lysine could be used to modulate degradation of the collagen (FIG. 4). The addition of TEOS reduced the rate of degradation for the collagen gels. Furthermore, aging of the nanocomposite hydrogels proved to be significant for reducing degradation rate. The tests were concluded when the gels became too weak to handle. The differences in residual weight percentage between the control, TA2-UV4, and TA10-UV10 at times above 200 minutes are statistically significant. The gel that was aged for 10 days in TEOS/ethanol and exposed to UV for four hours maintained mechanical stability for twice as long as the control and over 200 minutes longer than the sample that was only aged for two days in TEOS/ethanol.

### Spectral Transmittance

The 50 mg/mL control (FIG. 5) had a reduced spectral transmittance at lower wavelengths compared to previously published data for an excised rabbit cornea (McLaren J W and Brubaker R F, 1996. Curr Eye Res 15(4):411-421). The spectral transmittance does not change significantly after silica deposition, aging, and/or UV exposure, however.

### Mechanical Properties

The addition of silica resulted in an increase of stiffness for three different collagen concentrations. For 5 mg/mL, the control sample was too weak to handle and a significant increase in stiffness occurred from soaking in poly-L-lysine and addition of LUDOX silica spheres (W. R. Grace & Co., Columbia, Md.). The stiffness continued to increase as the silica spheres were cross-linked with TEOS. For 50 mg/mL gels, the stiffness increased by approximately two orders of magnitude after silica deposition, aging and UV exposure. While the UV exposure alone contributed to the stiffening of collagen fibrils, the difference between the UV and TA10-UV4 relaxed modulus is statistically significant. The 100 mg/mL gels followed the same trend.

This disclosure therefore describes a novel method that produces silica-collagen hydrogels that have properties advantageous for use as an artificial corneal transplant material. The combination of treating the fibrillar component (e.g., collagen network) with a cationic component (e.g., poly-L-lysine) and a Stöber soaking resulted in uniform deposition of silica onto the fibrils. Poly-L-lysine has been used in layer-by-layer coatings with silica in past work (Zhu et al., 2004. Biotechnol. Appl. Biochem. 39(2): 179-187). Cationic poly-L-lysine gives the collagen fibril surface a uniform positive charge at neutral conditions. The hydrogel was then soaked in a silica precursor solution containing hydrolyzed TEOS molecules that have negative charge, which are electrostatically attracted to the positively charged collagen fibril surface.

In contrast, without pretreating the collagen surface with poly-L-lysine, aggregation between silica particles is more favorable than silica deposition at the collagen surface (FIG. 1C, FIG. 1E).

When using Stöber solutions, one can control the H₂O:Si molar ratio, R, to control the physical properties of the resulting material. FIG. 1E shows that R can be kept above 400 to avoid micron-size silica clusters. Using a more dilute solution of the silica precursor increases the likelihood that polymerization takes place at the collagen surface, thereby limiting the particle sizes and light scattering. Additionally, FIG. 2 shows that this deposition technique can be used on highly concentrated hydrogels, resulting in a uniform increase in fibril diameter without a reduction in spectral transmittance (FIG. 5).

Aging and UV exposure can reduce the degradation rate of the gels. Gels that were not aged showed only a slight increase in degradation resistance. From the Raman spectra, one can see from the intensity increase in the —CH peak region that the silica network formed around the collagen is not highly condensed and still maintains some of its alkyl side chains. This is reduced after aging and densifying the network with UV exposure, which results in a significant reduction in degradation rate (FIG. 4). The dissolution of silica is reduced by further increasing the extent of condensation and reducing the number of silanol (Si—OH) groups present in the gel. The aging and densification methods described in this disclosure are one way of doing this. Alternative modifications of the silica network also are possible. For example, dipodal silanes have been used to improve the hydrolytic stability of silica gels. Also, PEG-silanes with various chemistries can be used to change the behavior of the silica networks (vary porosity, hydrophobicity, act as cross-linker between silanol groups).

The mechanical stiffness of gels was increased upon silica deposition, aging, and UV exposure. For 5 mg/mL gels, adding LUDOX spheres (W. R. Grace & Co., Columbia, Md.) improved the mechanical properties of the gels, causing them to transition from weak gels that did not maintain their shape to gels that can be handled and manipulated without failure. This result was further exaggerated for longer Stöber soaking times. For higher concentrations of collagen, the mechanical behavior began to approach that of the native cornea with stiffness values of approximately 1 MPa. While it is difficult to make comparisons due to differences in testing protocols, corneal stiffness values of 0.8-5 MPa have been reported for strains ranging from 4-10%. For strains above 10%, the stiffness of the cornea has been estimated to be 35-60 MPa. These previous tests did not allow for the material to relax, however, and often used high strain rates. Because of the viscoelastic behavior of collagenous tissue, equilibrium stiffness values are often overestimated when higher strain rates are used.

This disclosure describes a two-step method for silica-deposition onto fibrils of a biocompatible polymer. The method promotes formation of fibrils that exhibit increased stiffness and/or reduced enzymatic degradation rate of, for example, collagen hydrogels without reducing the visible light spectral transmittance. Chemical characterization via Raman spectroscopy and in vitro degradation experiments showed that aging and densification of the deposited silica resulted in a reduction of degradation rate.

In another aspect, this disclosure therefore describes a biocomposite material suitable for use in, for example, an ocular implant. Generally, the biocomposite material includes a fibril that includes a biocompatible polymer and a cationic component, and a uniform coating of silica-containing material.

While described herein in the context of exemplary embodiments in which the fibril includes collagen, the biocompatible material can include alternative fibrillar biocompatible materials either in place of or in addition to collagen. The fibril can, therefore, include any of the fibrillar polymeric structural proteins of the body such as, for example, a collagen, an elastin, keratin, actin, and/or myosin.

Also, while described herein in the context of an exemplary embodiment in which the cationic component includes poly-L-lysine, the cationic component can includes alternative cationic materials either in place of or in addition to poly-L-lysine. The fibril can therefore include, as a cationic component, a surfactant (e.g., polyethylene oxide, polypropylene oxide, lysine, polydimethylsiloxane (PDMS), cetrimonium bromide (CTAB), polyvinylpyrrolidone (PVP), dodecyldimethylethylammonium bromide (DDAB), cetyltrimethylammonium hydroxide (CTAOH), gelatin, polyethylene glycol (PEG)), one or more sugars (e.g., fructose) linked to the biocompatible polymer (e.g., via a Maillard reaction, and/or positively-charged collagen. Collagen may be prepared under acidic conditions so that the collagen is positively charged. The silica component may be applied to the fibril while the fibril retains the positively charged collagen.

Silica is naturally occurring and biocompatible. While described herein in the context of exemplary embodiments in which the silica-containing material includes tetraethylorthosilicate (TEOS), the silica-containing material used to form the coating can be formed from any suitable silica precursor. Exemplary silica precursors include those shown in Table 1. The biocompatible composite material can include any, or any combination of two or more, of the silica precursors listed in Table 1.

TABLE 1
Exemplary Silica Precursors

| Chemical Name | Acronym | Molecular Formula |
|---|---|---|
| Tetramethylorthosilicate | TMOS | Si(OCH₃)₄ |
| Tetraethylorthosilicate | TEOS | Si(OC₂H₅)₄ |
| Tetrakis(2-hydroxyethyl)orthosilicate | THEOS | Si(OCH₂CH₂OH)₄ |
| Methyldiethoxysilane | MDES | C₅H₁₄O₂Si |
| 3-(Glycidoxypropyl)triethoxysilane¹ | GPTES | C₁₂H₂₆O₅Si |
| 3-(Glycidoxypropyl)trimethoxysilane¹ | GPTMS | C₉H₂₀O₅Si |
| 3-(Trimethoxysilyl)propylacrylate | TMSPA | H₂C═CHCO₂(CH₂)₃Si(OCH₃)₃ |
| N-(3-Triethoxysilylpropyl)pyrrole | TESPP | |
| Vinyltriethoxysilane | VTES | H₂C═CHSi(OC₂H₅)₃ |
| Vinyltrimethoxysilane | VTMES | H₂C═CHSi(OCH₃)₃ |
| Methacryloxypropyltriethoxysilane | TESPM | |
| Silica Nanoparticles^{1,2} | | SiO₂ |
| Sodium Silicate (e.g., 27% Silicic Acid 10% NaOH) | Water glass | |
| Diglycerylsilane¹ | DGS | |
| Methyltriethoxysilane¹ | MTMOS | CH₃Si(OCH₃)₃ |
| 3-aminopropyltriethoxysilane¹ | APTS | H₂N(CH₂)₃Si(OC₂H₅)₃ |
| 3-aminopropyltrimethoxysilane¹ | APTMS | C₆H₁₇NO₃Si |
| 3-(2,4-Dinitrophenylamino)propyltriethoxysilane | | |
| Mercaptopropyltriethoxysilane | TEPMS | HS(CH₂)₃Si(OCH₂CH₃)₃ |
| 3-(2-Aminoethylamino)propyltriethoxysilane | | (CH₃O)₃Si(CH₂)₃NHCH₂CH₂NH₂ |
| Isocyanatopropyltriethoxysilane¹ | | C₁₀H₂₁NO₄Si |
| Hydroxyl-terminated polydimethylsiloxane¹ | PDMS | |
| Triethoxysilyl-terminated polydimethylsiloxane¹ | PDMS | |
| Methyltriethoxysilane¹ | MTES | CH₃Si(OC₂H₅)₃ |
| Triethoxysilyl-terminated poly(oxypropylene) | | |

¹Exemplary precursors that may function as plasticizers.
²e.g., LUDOX (W. R. Grace & Co., Columbia, MD), NYACOL (Nyacol nano Technologies, Inc., Ashland, MA), or CAB-O-SIL (Cabot Corp., Boston, MA)

The two-step method described herein produces a biocompatible composite that possesses a more uniform silica coating than is possible using other methods. As shown in FIG. 1 and FIG. 2, biocompatible composites produced as described herein possess fewer and smaller silica aggregates compared to comparable conventionally-produced composites. In some embodiments, a measure of the uniformity of the coating can include reference to the largest diameter silica aggregate in the coating. In some of these embodiments, the coating can include no silica aggregates greater than 100 nm in diameter. Specifically, FIG. 1 shows the formation of a biocompatible composite using a 5 mg/ml gel and having silica particles no greater than about 100 nm in diameter. Deposition of the silica particles on the fibril can be enhanced with the use of poly-L-lysine or other cationic material applied to make the surface of the fibril positively charged. FIG. 2 shows the formation of a biocompatible composite using a 50 mg/ml gel and having silica particles no greater than about 15 nm in diameter. In this embodiment, the silica particles again interact only with the underlying biopolymer fibril and not with other silica particles. Thus, one can to a certain degree control the size of the silica particles in the coating.

Accordingly, in some embodiments, the coating can possess silica nanoparticles having a maximum diameter of no greater than 500 nm such as, for example, no greater than 450 nm, no greater than 400 nm, no greater than 350 nm, no greater than 300 nm, no greater than 250 nm, no greater than 200 nm, no greater than 150 nm, no greater than 100 nm, no greater than 90 nm, no greater than 80 nm, no greater than 70 nm, no greater than 60 nm, no greater than 50 nm, no greater than 40 nm, no greater than 30 nm, no greater than 25 nm, no greater than 20 nm, no greater than 15 nm, or no greater than 10 nm.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Materials

Soluble type I collagen from rat tail tendon was purchased from Alphabioregen (Worcester, Mass.) at a stock concentration of 5 mg/mL and a pH of 3. Cellulose dialysis membrane rated for a molecular weight of 12 kDa, poly-L-lysine (PLL) (1-5 kDa), reagent grade tetraethyl orthosilicate (TEOS) (98%), LUDOX SM (30% w/w; W. R. Grace & Co., Columbia, Md.), ammonium hydroxide (28-30% w/w), glutaraldehyde solution (25%), osmium tetroxide (4% in water), and collagenase (type I from clostridium histolyticum) were purchased from Sigma-Aldrich (St. Louis, Mo.). Polyethylene glycol (PEG) with a molecular weight of 35 kDa and sucrose were purchased from EMD Millipore (Billerica, Mass.). Sodium cacodylate trihydrate was purchased from Electron Microscopy Sciences (Hatfield, Pa.).

### Collagen Hydrogel Synthesis

A stock collagen solution of soluble type I rat tail collagen was used as the main scaffold component. In some cases, the concentration was increased using a simple dialysis process. Briefly, a cellulose dialysis membrane was soaked in deionized (DI) water, and collagen was poured into one end of the tubing while the opposing end was clamped shut by a dialysis clip. The open end was sealed with another dialysis clip. The dialysis bag containing collagen was then dialyzed against a 0.1 g/mL PEG solution at 4° C. until concentrations between 10 mg/mL and 100 mg/mL were reached. Collagen concentration was estimated by the volumetric water loss of the collagen solution.

A small volume of the concentrated collagen solution was placed on a glass slide, and rubber spacers were used to create a film with a uniform thickness of 0.5 mm (roughly the thickness of the stroma in the native cornea). The film was then exposed to ammonia vapor in an enclosed petri dish for ten minutes to induce fibril formation. The collagen gels were rinsed with DI water, and soaked in a 10 μg/mL poly-L-lysine solution for 1 hour at room temperature.

### Silica Deposition

Prior to soaking collagen hydrogels in a Stöber solution, a solvent exchange was performed. Hydrogels were sequentially soaked in 20%, 40%, 60%, and 80% ethanol baths for 10 minutes each. These samples were then placed in an ethanol/water/TEOS/ammonium hydroxide solution. Silica precursor solutions for single hydrogel discs (5 mm diameter) were prepared in the following manner. TEOS, 200 proof ethanol, and DI water were mixed vigorously. This Stöber solution had an H₂O:Si molar ratio (R) of 40,000 and a 4:1 ethanol:H₂O volumetric ratio. This R value was used for all gels unless stated otherwise. Ammonium hydroxide was added dropwise until the pH of the solution was approximately 9. The poly-L-lysine treated hydrogel was then placed in the Stöber solution and gently agitated. After 24 hours of soaking, the Stöber solution was replaced with a fresh solution. For some 5 mg/mL hydrogels, a one hour soak in a stable LUDOX silica sphere suspension (3% w/w; W. R. Grace & Co., Columbia, Md.) was performed between the poly-L-lysine treatment and Stöber soaking steps.

### Silica-Collagen Aging/Densification

Aging methods were used to maximize the condensation of silica networks formed during deposition. The methods used were similar to those used by Hæreid et al. for TEOS alcogels (Hæreid et al., 1995. J. Non. Cryst. Solids 186:96-103). Briefly, after silica deposition each sample was put in a solution consisting of 1 mL of 80% ethanol and 30 μL of ammonium hydroxide for 24 hours. Then, these samples were kept in a TEOS/ethanol solution (7:3 volume ratio) for different periods of time.

In an attempt to densify this network and maximize condensation of the deposited silica, the aged gels were exposed to ultraviolet radiation (UV) by placing hydrated gels in the center of a sterile laminar flow hood with a 39 W UV light source (Imai et al., 1999. Thin Solid Films 351(1):91-94). The effects from time of UV exposure were examined. For simplicity and brevity, the abbreviations listed in Table 2 are used in all subsequent results.

TABLE 2
Sample labels used in the following sections that correspond to the final
step of each hydrogel treatment

| Sample Label | Sample Description |
|---|---|
| Control | Collagen Hydrogel |
| +PLL | After PLL addition |
| +TEOS | After 5 day Stöber soak |
| | After 1 day NH₄OH/Ethanol soak |
| TAn | After n day TEOS/Ethanol soak |
| TAn-UVm | After m hour UV exposure |

PLL: poly-L-lysine
TEOS: tetraethyl orthosilicate

### Scanning Electron Microscopy (SEM)

Hydrogel samples were fixed according to standard methods. Briefly, samples were soaked in a 2% glutaraldehyde solution containing 0.1 M sucrose and 0.1 M sodium cacodylate for one hour at room temperature. The samples were then post fixed in a 1% osmium tetroxide solution for 30 minutes, after which they were sequentially soaked in 20%, 40%, 60%, 80%, 95%, and 100% ethanol solutions for ten minutes each. Samples were critical-point dried with a Tousimis samdri-780A CO₂ critical point dryer (Tousimis, Rockville, Md.), attached to SEM stubs with carbon tape, and coated with 5 nm of Platinum. Images were obtained using a JEOL 6500 SEM (JEOL USA, Peabody, Mass.) with a 5 kV beam.

### Raman Spectroscopy

Raman samples were not fixed, but were critical-point dried immediately after silica deposition and aging/densification. Raman spectra of the samples were collected with a Witec (Ulm, Germany) alpha300 R confocal Raman microscope equipped with a UHTS spectrometer and DV401 CCD detector. A 10 mW Nd:YAG laser was used as an excitation source and focused on the sample with a Nikon 10× air objective (Melville, N.Y.). Spectra collection consisted of 20 accumulations each with a 30 second integration time. The spectra was collected and processed using the Witec control software.

### Collagenase Degradation Assay

The resistance to enzymatic degradation was quantified using an in vitro assay with bacteria-derived collagenase. An enzyme solution was prepared by dissolving lyophilized collagenase in a phosphate buffer solution (PBS) at a concentration of 10 units/mL. The solution was preheated to 37° C. in a petri dish. For every sample (0.5 mm thick, 20 mm² area), 0.5 mL of enzyme solution was added. The samples were incubated at 37° C., and the weight of each sample was measured hourly until the sample became too weak to handle.

### Optical Characterization

The spectral transmittance of samples was measured using a spectrophotometer (SpectroMax Plus; Molecular Devices, Sunnyvale, Calif.). The samples were placed on a glass slide and held in place by a rubber mold and covered by a glass coverslip. The glass slide was placed vertically in the cuvette chamber and perpendicular to the incident beam and light sensor. The transmittance was then measured in 100 nm wavelength increments from 300 to 800 nm. These transmittance values were divided by the transmittance of a PBS blank to adjust for light scattering associated with the glass slide holder.

### Mechanical Characterization

Hydrogel strip specimens (see collagen hydrogel synthesis above) were 0.5 mm thick and approximately 3 mm wide. Mechanical tests were performed at the University of Minnesota Tissue Mechanics Lab using a low force tensile tester (Instron, Norwood, Mass.). A PBS bath was used to maintain sample hydration for the duration of the experiments. The sample ends were loaded into two opposing grips with a constant gauge length of 3 mm. The specimens were preconditioned with ten cycles of a sinusoidal strain (amplitude=5% strain, wavelength=4 seconds). Following preconditioning, the strain was ramped at rates of 1% per second to 8, 18, and 30%, respectively. At each step strain, the material was allowed to relax for three minutes. The stresses after the three minutes of relaxation were plotted versus strain, and the slope of this curve was defined as the relaxed modulus of the material.

### Statistics

In order to quantify the statistical significance of the data reported, all nominal values were reported with a confidence limit which is defined as the uncertainty associated with an estimated mean. The following formula was used to calculate the confidence limit of the mean for a given sample,

$$Confidence Limit = mean value \pm \text{?}$$

$$\text{?} \text{indicates text missing or illegible when filed}$$

where t is the two-sided t-distribution value for a given number of data points N and confidence coefficient α, and s is the standard deviation of the sample. A confidence coefficient of 95% was used when calculating the confidence limit. If the confidence limit of one sample did not overlap the confidence limit of another sample, the difference in mean values of these two samples was deemed statistically significant.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method of making a biocompatible composite, the method comprising:
forming a fibril comprising a hydrogel that comprise a biocompatible polymer;
treating the fibril with a cationic component; and
coating the fibril with a silica-containing component.

2. The method of claim 1 wherein the biocompatible polymer comprises collagen.

3. The method of claim 2 wherein the silica-containing component forms a uniform coating on the surface of the fibril.

4. The method of claim 3 wherein the uniform coating comprises silica aggregates of no more than 100 nm in diameter.

5. The method of claim 1 wherein the cationic component comprises poly-L-lysine.

6. The method of claim 1 wherein the silica-containing component forms a uniform coating on the surface of the fibril.

7. The method of claim 6 wherein the uniform coating comprises silica aggregates of no more than 100 nm in diameter.

8. A biocompatible composite comprising:
a fibril comprising a biocompatible polymer and a cationic component; and
a uniform coating of silica-containing material.

9. The biocompatible composite of claim 8 wherein the cationic component comprises poly-L-lysine.

10. The biocompatible composite of claim 9 wherein the uniform coating comprises no silica aggregates greater than 100 nm in diameter.

11. The biocompatible composite of claim 9 wherein the biocompatible polymer comprises collagen.

12. The biocompatible polymer of claim 11 wherein the silica-containing component comprises tetraethylorthosilicate (TEOS).

13. The biocompatible composite of claim 8 wherein the biocompatible polymer comprises collagen.

14. The biocompatible polymer of claim 13 wherein the silica-containing component comprises tetraethylorthosilicate (TEOS).
